(19) United States
(12) Patent Application Publication
(10) Pub. No.: US 2022/0031162 A1
(43) Pub. Date: Feb. 3, 2022

(54) STROKE DETECTION AND MITIGATION

(71) Applicant: Healthcare Integrated Technologies Inc., Knoxville, TN (US)

(72) Inventors: Kenneth M. GREENWOOD, Davenport, FL (US); Scott Michael BORUFF, Knoxville, TN (US); Jurgen VOLLRATH, Sherwood, OR (US)

(21) Appl. No.: 17/392,033

(22) Filed: Aug. 2, 2021

Related U.S. Application Data
(60) Provisional application No. 63/103,410, filed on Aug. 3, 2020.

(51) Int. Cl. A61B 5/00; G06K 9/00; G10L 25/78; G10L 15/22

(52) U.S. Cl. CPC A61B 5/0022; A61B 5/746; G06K 9/00348; G06K 9/00268; G06K 2009/00738; G10L 25/78; G10L 15/22; A61B 5/4806; G06K 9/00711

(57) ABSTRACT

A method and system for detecting a possible stroke in a person through the analysis of voice data and image data regarding the gate of the user, facial features and routines, and corroborating any anomalies in one set of data against anomalies in another set of data for a related time frame.

### FIELD OF THE INVENTION

The invention relates to home care. In particular it relates to detecting when a person in the home is experiencing a stroke.

### BACKGROUND OF THE INVENTION

A major health issue that has become particularly prevalent in the last 50 years is the incidence of stroke, which can leave people with permanent disability. Even though many of the effects of a stroke could be reversed if caught early, the diagnosis or treatment often take place outside the critical window of opportunity for reversing the effects of stroke.

The present invention seeks to address this problem.

### SUMMARY OF THE INVENTION

The present invention proposes a system and method of identifying and intervening in a stroke quickly, thereby giving the patient (also referred to herein as the user) a much better chance of recovery or with much fewer or milder permanent symptoms.

According to the invention there is provide a system that includes one or more image sensor for capturing the gate of a person in a building. In most circumstances the building will be the apartment or home of the user. The image sensor may be a video camera or radar detector, lidar or radio frequency image sensor for capturing the gate of a user.

An algorithm, which may be implemented as an artificial intelligence (AI) system may be configured to identify anomalies in the user's gate e.g. tumbling, instability, wobbling, or problems with coordination.

Detection of an anomaly by the one or more image sensors may trigger a video camera (which may form part of the one or more image sensors) to zoom in on the user based on the location of the user in the residence as determined by the image sensors or a separate movement detector, e.g. infra-red (IR) sensor. The image sensor may comprise an internet RGB camera with IR capabilities. The zoom in video camera may be a separate camera or form part of the image sensor, and may capture facial details to corroborate possible stroke diagnosis, e.g. facial muscle weakness or partial paralysis/drooping of parts of the user's face.

In addition, or alternatively the user's speech may be analyzed for anomalies, e.g. by engaging the user in conversation. In one embodiment, a voice-bot or other voice interactive platform engages the user in conversation for purposes of detecting speech anomalies indicative of a stroke: difficulty speaking, slurred speech, or speech loss, or the absence of a response. The voice interactive platform may for example ask the user “Are you ok?”, in response to detecting a problem in the user's gate, or it may already be engaged in conversation with the user and identify an anomaly in the user's speech that triggers the system to identify a possible stroke event.

The system may, upon a diagnosis of a potential stroke event propose immediate action by the user, e.g. having the voice-bot instruct the user: “Go to your medicine cabinet in the bathroom right now and take an aspirin.”

The system may also notify authorized persons, e.g. primary care physician, family member, care provider, etc., to have them check on or follow up with the user.

Thus, according to the invention, there is provided a method for detecting and mitigating the impact of a stroke in a user, comprising monitoring one or more of a user's gate patterns and speech patterns, detecting an anomaly in one or more of the gate patterns and speech patterns of a user based on previously stored data about one or both of the user's gate patterns and speech patterns, verifying an anomaly in the speech patterns by comparing the anomaly in the speech patterns with gate data captured during a related time-frame, or verifying an anomaly in the gate patterns with speech data captured during a related time-frame, and triggering a stroke event in the case of a verification.

The triggering of a stroke event (also referred to herein as a flagging event) may include at least one of instructing the user to take some form of action, and notifying one or more pre-defined persons.

The verifying may include capturing a third type of information additional to the speech pattern and gate pattern data, that may be used to verify a stroke event.

Further, according to the invention, there is provided a system for detecting a stroke in a user and mitigating the impact of a stroke, comprising a video camera for monitoring at least one of user gate, and user habits, a microphone and a speaker defining an interactive voice platform, a processor and memory configured with machine readable code defining an algorithm for analyzing data from the at least one video camera and the microphone to identify anomalies in any of the image data from the video camera or voice data from the microphone of the user indicative of a possible stroke, and for validating anomalies in the image data or speech data, with data from the microphone or video camera, respectively, to define a stroke event.

The anomalies in the image data may include anomalies in the user's gate, which may include one or more of: tumbling, instability, wobbling, and problems with coordination. The image data may also include close-up image data of the user's face to identify facial muscle weakness or partial paralysis/drooping of parts of the user's face

The anomalies in the voice data of the user may include one or more of: difficulty speaking, slurred speech, speech loss, and the absence of a response or non-sensical response when prompted via the speaker.

The system may further comprise a communications system for notifying one or more predefined persons in the event of a stroke event.

The system may also include a storage medium for storing at least one of speech patterns and gate patterns of the user, wherein the identifying of anomalies comprises comparing data from the video camera and microphone (collectively referred to as sensors) with the patterns previously stored for the user.

The storage medium may include data defining daily habits, including sleep patterns of the user, for identifying variations in the habits as captured by the sensors.

The system may include a server in communications with the sensors.

The processor may include at least one of: a local processor for local processing of sensor data, and a remote processor at the server or at a client device connected to the server.

The identifying and validating of anomalies by the processor is typically controlled by machine readable code on a memory or storage medium, and the machine-readable code may include logic to instruct the interactive voice platform in case of an anomaly, to prompt the user with questions via the speaker in order to gain additional voice data. The machine readable code may be configured to notify one or more of the pre-defined persons if a defined anomaly is detected by any of the sensors or a stroke event is identified.

### BRIEF DESCRIPTION OF THE DRAWINGS

FIG. 1 is a depiction of one embodiment of the implementation of a system of the invention;

FIG. 2 is a flow chart defining the logic of one embodiment of an anomaly detection algorithm implemented in an AI system, and

FIG. 3 is a flow chart defining the logic of one embodiment of an anomaly detection and corroboration algorithm implemented in an AI system.

### DETAILED DESCRIPTION OF THE INVENTION

The present invention is applicable to any surveilable locations, e.g. parking lots, malls, restaurants, etc., but for ease of description, and since people, especially the elderly, spend most of their time at home, the present invention will be described with respect to the home environment. This may be a user's house, apartment or temporary or permanent care facility.

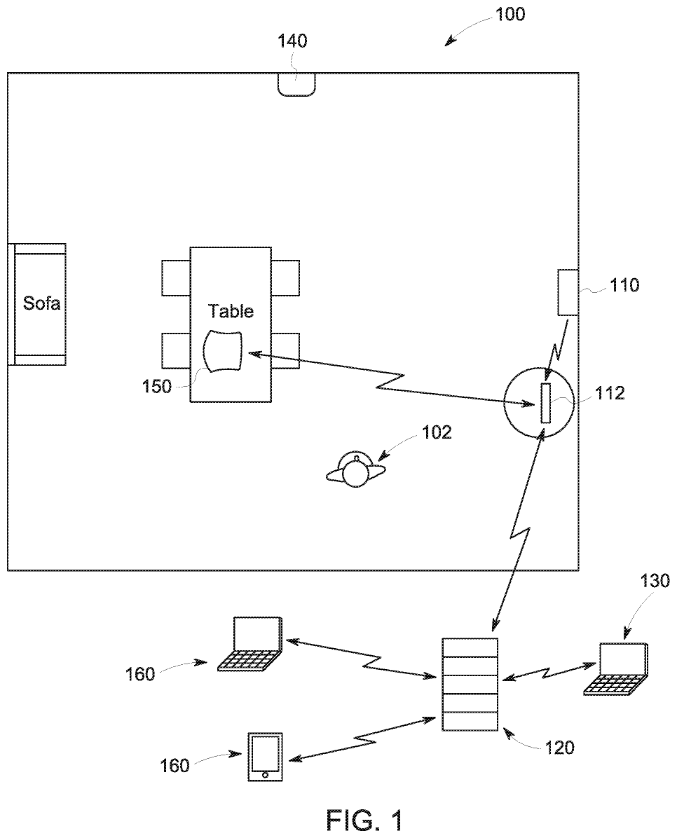

One such implementation is shown in FIG. 1, which shows a plan view of an apartment 100 with a user 102. An image sensor in the form of a video camera or non-invasive image sensor such as radio-frequency image sensor, lidar or radar 110 is mounted on one of the wall of the apartment 100 and monitors the activities and movements of the user 102 for any anomalies.

The image sensor 110 is connected to a server 120, which may comprise a dedicated remote server or a cloud server system, as depicted here. In the present embodiment, the image sensor 110 communicates via short range communication (in this case Bluetooth connection) with a communications hub 112, which in turn communicates data from the image sensor 110 to the server 120.

The benefit of using a video camera as the image sensor is that it provides higher resolution images and allows for more detailed analysis of the user. The image sensor 110 or hub 112 may include a processor (not shown) and memory with machine readable code for controlling the processor to perform some of the data processing locally, e.g. anomaly detection, or anomaly detection and analysis. These anomalies may comprise identifying changes in the user's routine. In particular, in the present application, the processor may specifically identify anomalies in the gate of the user e.g. tumbling, instability, wobbling, or problems with coordination. The analysis may be based on a comparison to previously recorded gate patterns of the user, which in one embodiment includes storing previous gate patterns in a memory e.g., in a database of gate patterns.

In another embodiment, some or all of the processing may be performed remotely, e.g. at the server 120 or at a client 130 connected to the server. For example, a local processor may identify anomalies and send only those image frames or data streams associated with the anomalies, while the analysis and comparison to previously stored gate patterns of the user or of third parties may be performed remotely.

In yet another embodiment, all of the data from the image sensor 110 is transmitted to the server120 via the hub 112 or directly from the image sensor 110 insofar as the image sensor includes a long distance communications system, e.g. cell phone transmitter, Wi-Fi, etc. In such a situation the data may be stored at the server 120 and processed at the server 120 or at a client 130 connected to the server 120 to identify a possible stroke event.

In the above embodiment, the identification of a possible stroke event was based on gate anomalies of the user. However, the present invention also includes the monitoring of other activities and habits of the user to identify a possible emergency event (also referred to herein as a flagging event or stroke event). For instance, the sleep patterns of a user may be monitored and compared to typical sleep pattern data that has been captured for the user. Departures from such routines by a predefined amount, e.g. remaining in bed more than half an hour after typical times that the user would get up, may indicate a possible stroke event. Similarly lying down by the user on the floor, or extended periods of immobility in a chair or couch during the day may be indicative of a stroke event, depending on the nature of the anomaly (e.g., duration that the person remains immobile, or location where they are lying down). By grading the degree of the anomaly relative to previously recorded activity patterns of the user, the anomaly may be elevated to an emergency (also referred to herein as a stroke event or flagging event). In another embodiment, or if the anomaly does not reach the level of an event, the system may initiate a validation or corroboration step, as discussed in more detail below.

In one embodiment of the present invention, the identification of a possible stroke event may be validated using one or more secondary sources of data.

In the embodiment proposed in FIG. 1, the image sensor 110 is a non-invasive image sensor with limited resolution. Therefore, it may be supplemented by a separate video camera 140, which is included as part of the system. The video camera 140, in this embodiment is mounted on a wall of the apartment 100 and is normally dormant, in order to protect the privacy of the user 102. In the event of an anomaly being detected by the image sensor 110 in conjunction with the local and/or remote processing activity, or in some embodiments only in the event of a possible stroke event being detected based on the analysis and comparison to previous gate data, an algorithm stored in memory associated with the local or remote processor may instruct the video camera to zoom in on the user 102 to capture facial features of the user. Locating of the user 102 may be based on data from any existing motion detector, e.g. infra-red (IR) detector, or may be based on data received from the image sensor 110.

The video camera 140 may capture facial details to corroborate a possible stroke diagnosis, e.g. facial muscle weakness or partial paralysis/drooping of parts of the user's face. If any of these features or any other features commonly associated with a stroke, are identified, the possible stroke event may be elevated to a confirmed event, which may trigger the system to initiate a follow-up response as is discussed in greater detail below.

In the present embodiment, the data from the image sensor 110, identifying an anomaly in the user's gate or any other anomaly indicative of a stroke event, may also rigger another form of corroboration, e.g., by analyzing vocal data from the user. In the present embodiment this is implemented in the form of a voice-bot 150. The voice-bot 150 may be implemented as a robot or may simply comprise software on a user's computing device, e.g. desktop or laptop, that supports interactive voice communication (collectively referred to herein as an interactive voice platform). In response to an anomaly being detected by the image sensor 110 or based on the analysis and comparison to previous gate data, the voice-bot 150 may engage the user 102 in conversation, e.g. by asking: “Are you alright?”. Based on the response received, or lack of a response, and based on the nature of the speech patterns of the user, e.g., difficulty speaking, slurred speech, or incoherent speech, the possible stroke event may be elevated by the system to the level of a confirmed stroke event (flagging event), thereby triggering a follow-up response.

The follow-up response in this embodiment comprises both an instruction to the user and notification of authorized persons.

While the above example described the triggering event being an anomaly detected by the image sensor 110, while the video camera 140 and voice-bot 150 acted as corroborating devices, it will be appreciated that the triggering may be initiated, for example, by the voice-bot with corroboration by the image sensor 110 and video camera 140. For instance, the user 102 may be in communication with the voice-bot, when the voice-bot detects an anomaly in the speech of the user. The voice communications between user and voice-bot may be uploaded to the server where a processor analyses the speech patterns of the user for any anomalies, which then trigger corroboration by the video camera 140 and/or by analysis of the data from the image sensor 110.

It will be appreciated that the corroborating data from one or more sensors or devices (image sensor, video camera, voice-bot) has to be captured during the same time-frame or closely adjacent time-frame (referred to herein generally as a related time-frame) as the triggering sensor or device, and for a long enough period to allow the data from the sensors/devices to be compared with respect to the same event.

The follow-up response mentioned above may comprise an instruction to the user. This may take the form of a prompt by the server 120 via the hub 112, instructing the voice-bot 150 to enter an alarm mode, which in this embodiment causes the voice-bot 150 to issue a voice instruction to the user, e.g.: “Go to your medicine cabinet right now and take an aspirin.”

The server 120 also notifies a family member, primary care physician, care-giver and/or other person (collectively referred to herein as responders) that the user may be experiencing a stroke, thereby insuring that other persons apart from the user can rapidly intervene and see to the user's well-being. This communication may comprise initiating a call to a cell phone number, or generation of a text message, to one or more numbers on file, associated with the responders.

In one embodiment, the system may include an interactive platform that users and responders can access using client devices 160. Such an interactive platform may be implemented as a dashboard that is optionally voice-activated and which is accessible using a client device 160 browser or via a downloadable software application (App). The interactive platform may grant different responders different levels of access, e.g. family members may be given access to feed from the video camera when an anomaly is detected, while other responders may not be provided with such personal data or may only receive it once an event is confirmed. In such an embodiment where there is an interactive platform, a notification to responders may take the form of an emergency notice (visual and/or audible alarm) sent to a client device 160.

The implementation of an anomaly analysis as discussed above, requires logic in the form of machine readable code defining an algorithm or implemented in an artificial intelligence (AI) system, which is stored on a local or remote memory (as discussed above), and which defines the logic used by a processor to perform the analysis and make assessments.

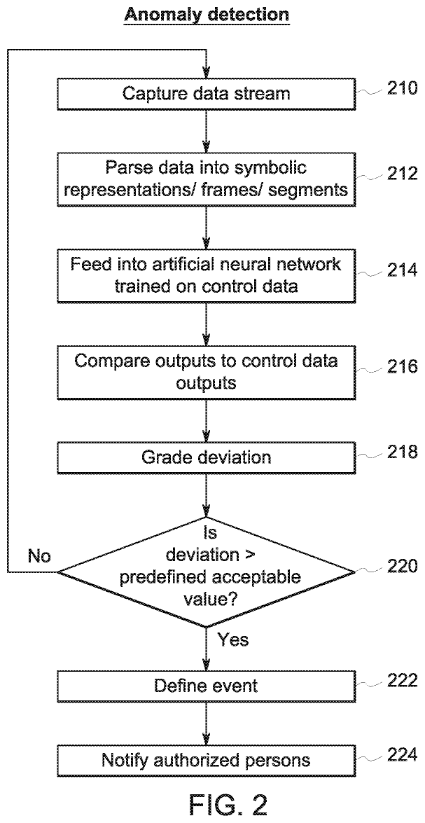

One such embodiment of the logic based on grading the level of the anomaly, is shown in FIG. 2, which defines the analysis based on sensor data that is evaluated by an Artificial Intelligence (AI) system, in this case an artificial neural network. Data from a sensor is captured (step 210) and is parsed into segments (also referred to as symbolic representations or frames) (step 212). The symbolic representations are fed into an artificial neural network (step 214), which has been trained based on control data (e.g. similar previous events involving the same party or parties or similar third-party events). Examples of symbol representations include anomalies in the user's gate e.g. tumbling, instability, wobbling, or problems with coordination, facial muscle weakness or partial paralysis/drooping of parts of the user's face.

The outputs from the AI are compared to outputs from the control data (step 216) and the degree of deviation is graded in step 218 by assigning a grading number to the degree of deviation. In step 220 a determination is made whether the deviation exceeds a predefined threshold, in which case the anomaly is registered as an event (step 222) and one or more authorized persons is notified (step 224)

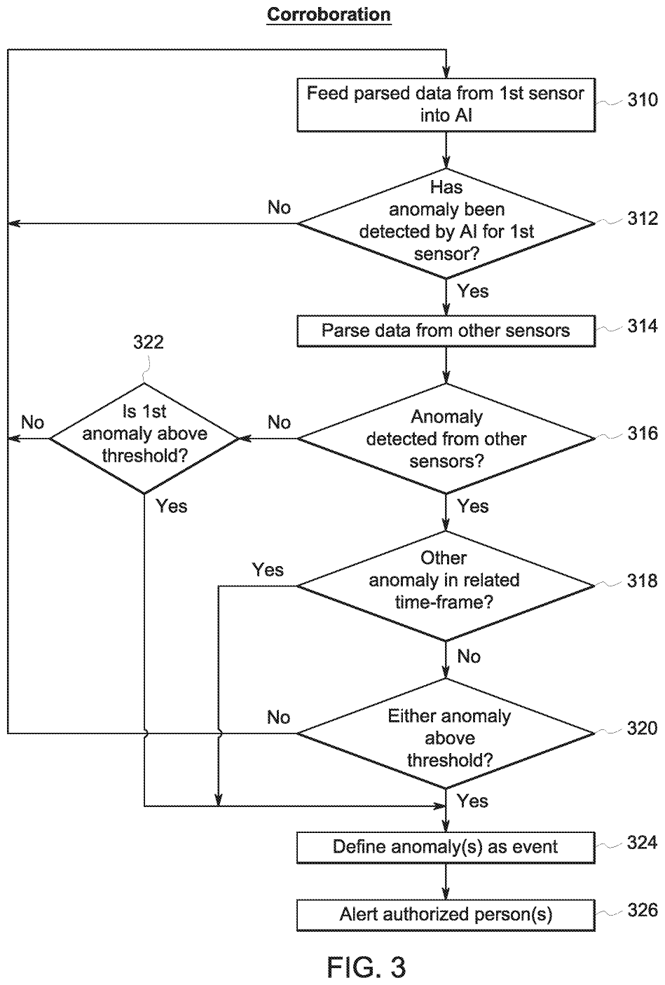

Another embodiment of the logic in making a determination, in this case, based on grading of an anomaly and/or corroboration between sensors is shown in FIG. 3.

Parsed data from a first sensor is fed into an AI system (step 310). Insofar as an anomaly is detected in the data (step 312), this is corroborated against data from at least one other sensor by parsing data from the other sensors that are involved in the particular implementation (step 314). In step 316 a decision is made whether any of the other sensor data shows up an anomaly, in which case it is compared on a time scale whether the second anomaly is in a related time frame (which could be the same time as the first sensor anomaly or be causally linked to activities flowing from the first sensor anomaly) (step 318). If the second sensor anomaly is above a certain threshold deviation (step 320) or, similarly, even if there is no other corroborating sensor data, if the anomaly from the first sensor data exceeds a threshold deviation (step 322), the anomaly captured from either of such devices triggers an event (step 324), which alerts one or more authorized persons (step 326) or advises the user to take some action, e.g. “Go to the medicine cabinet and take an aspirin.”

While the present invention has been described with reference to particular embodiments with specific sensor embodiments, it will be appreciated that different sensors and different configurations of the communication systems and server system can be implemented without departing from the scope of the invention.

What is claimed is:

1. A system for detecting a stroke in a user and mitigating the impact of a stroke, comprising
a video camera for monitoring any two or more of a user's gate, a user's facial features, and a user's routines (collectively referred to herein as image parameters),
a microphone for monitoring a user's voice parameters, wherein the video camera and microphone are collectively referred to as sensors,
a processor, and
a memory configured with machine-readable code defining an algorithm for analyzing image data from the video camera and voice data from the microphone to identify anomalies in any of the image parameters or voice parameters indicative of a possible stroke, and for validating any anomaly in the data from the video camera or microphone by comparing said anomaly with any anomaly detected in any of the other parameters, to define a stroke event.

2. The system of claim 1, wherein the anomalies in the user's gate, include one or more of: tumbling, instability, wobbling, and problems with coordination.

3. The system of claim 1, wherein anomalies in the user's facial features include facial muscle weakness or partial paralysis/drooping of parts of the user's face.

4. The system of claim 1, wherein anomalies in the voice data of the user include one or more of: difficulty speaking, slurred speech, speech loss, and the absence of a response or non-sensical response when prompted via the speaker.

5. The system of claim 1, further comprising a communications system for notifying one or more predefined persons in the event of a stroke event.

6. The system of claim 1, further comprising a storage medium for storing speech patterns and one or more of gate patterns, routines and facial features of the user, wherein the identifying of anomalies comprises comparing data from the video camera and microphone with the patterns previously stored for the user.

7. The system of claim 1, wherein routines of the user comprise daily routines, including sleep patterns of the user.

8. The system of claim 1, further comprising a server in communications with the sensors.

9. The system of claim 1, wherein the processor includes at least one of: a local processor for local processing of sensor data, and a remote processor at the server or at a client device connected to the server.

10. The system of claim 5, further comprising a speaker forming an interactive voice platform with the microphone, wherein the machine-readable code defines an algorithm to instruct the interactive voice platform in the case of an anomaly identified in the data from any of the sensors, to prompt the user with questions via the speaker in order to gain additional voice parameters.

11. The system of claim 10, wherein the machine-readable code includes logic to perform at least one of: notifying one or more of the pre-defined persons via the communications system, and instructing the user via the speaker to take some form of action, if a defined anomaly is detected by any of the sensors or a stroke event is identified.

12. A system for detecting a stroke in a user and mitigating the impact of a stroke, comprising
a video camera for monitoring at least one of a user's gate, a user's facial features, and a user's routines,
a microphone and a speaker defining an interactive voice platform, wherein the video camera and microphone collectively are referred to as sensors,
a processor, and
a memory configured with machine-readable code defining an algorithm for analyzing data from the video camera and the microphone to identify anomalies in any of the image data from the video camera or voice data from the microphone indicative of a possible stroke in the user, and for validating anomalies in the image data or speech data, with data from the microphone or video camera, respectively, to define a stroke event.

13. The system of claim 12, wherein the anomalies in the image data include anomalies in the user's gate, comprising one or more of: tumbling, instability, wobbling, and problems with coordination.

14. The system of claim 12, wherein anomalies in the user's facial features include facial muscle weakness or partial paralysis/drooping of parts of the user's face.

15. The system of claim 12, wherein anomalies in the voice data of the user include one or more of: difficulty speaking, slurred speech, speech loss, and the absence of a response or non-sensical response when prompted via the speaker.

16. The system of claim 12, further comprising a communications system for notifying one or more predefined persons in the event of a stroke event.

17. The system of claim 12, further comprising a storage medium for storing speech patterns and one or more of gate patterns, routines and facial features of the user, wherein the identifying of anomalies comprises comparing data from the video camera and microphone with the patterns previously stored for the user.

18. The system of claim 12, wherein routines of the user comprise daily routines, including sleep patterns of the user.

19. The system of claim 12, further comprising a server in communications with the sensors.

20. The system of claim 12, wherein the processor includes at least one of: a local processor for local processing of sensor data, and a remote processor at the server or at a client device connected to the server.

21. The system of claim 12, wherein the machine-readable code defines an algorithm to instruct the interactive voice platform in the case of an anomaly identified in the data from any of the sensors, to prompt the user with questions via the speaker in order to gain additional voice data.

22. The system of claim 16, wherein the machine-readable code includes logic to perform at least one of: notifying one or more of the pre-defined persons, and instructing the user via the speaker to take some form of action, if a defined anomaly is detected by any of the sensors or a stroke event is identified.

23. A method for detecting and mitigating the impact of a stroke in a user, comprising
monitoring any two or more of a user's gate patterns, facial features, routines, and speech patterns (collectively referred to herein as user parameters),
detecting an anomaly in one or more of the user parameters based on previously stored data on two or more of the user's gate, facial features, routines, and speech,
verifying an anomaly in any one of the user parameters by comparing the anomaly with an anomaly in any one of the other user parameters captured during a related time-frame, and
triggering a stroke event in the case of a verification.

24. The method of claim 23, wherein the triggering of a stroke event (also referred to herein as a flagging event) includes at least one of instructing the user to take some form of action, and notifying one or more pre-defined persons.

25. The method of claim 23, wherein anomalies in the user's gate patterns, include one or more of: tumbling, instability, wobbling, and problems with coordination.

26. The method of claim 23, wherein anomalies in the user's facial features include facial muscle weakness or partial paralysis/drooping of parts of the user's face.

27. The method of claim 23, wherein anomalies in the user's speech patterns include one or more of: difficulty speaking, slurred speech, speech loss, and the absence of a response or non-sensical response when prompted via a speaker.
